(19) United States
(12) Patent Application Publication
(10) Pub. No.: US 2015/0094724 A1
(43) Pub. Date: Apr. 2, 2015

(54) JOINT SCRUBBER

(71) Applicant: Exsomed Holding Company LLC, Scottsdale, AZ (US)

(72) Inventors: Lloyd P. Champagne, Phoenix, AZ (US); Jozef Zoldos, Phoenix, AZ (US)

(21) Appl. No.: 14/503,119

(22) Filed: Sep. 30, 2014

Related U.S. Application Data
(60) Provisional application No. 61/885,651, filed on Oct. 2, 2013.

(51) Int. Cl. A61B 17/16

(52) U.S. Cl. CPC A61B 17/16; A61B 17/1659; A61B 2017/00862
USPC 606/79

(57) ABSTRACT

A medical device suitable for scrubbing a joint, such as a distal interphalangeal (DIP) joint, is disclosed. The device may include a first end, a second end, and a space there between, and a joint scrubber having a first end coupled to the second end of the arm. The joint scrubber is configured to break apart or loosen material in a joint in order to facilitate the insertion of another device, such as a bone screw.

### FIELD OF THE DISCLOSURE

The present disclosure generally relates to medical devices. More particularly, the disclosure relates to apparatus for treatment of joints and to methods of using the same.

### BACKGROUND OF THE DISCLOSURE

Arthritis is a disease that affects the joints of a musculoskeletal system. The disease can cause pain, disfigurement, or worse if left untreated. A treatment for arthritis may depend of the form of arthritis, the joint(s) affected by the disease, and the severity of the disease.

When arthritis affects small joints, such as the distal interphalangeal (DIP) joint, the disease may be treated using a variety of techniques. When DIP arthritis is severe, for example, when pain associated with the disease cannot adequately be treated with medication, surgery can be used to treat the joint. In this case, joint fusion or arthrodesis, can be used to fuse adjacent bones together to alleviate some of the pain, discomfort, and/or disfigurement associated with DIP arthritis.

A typical surgical procedure to treat DIP arthritis includes open surgical resection of the joint to be treated and stabilization of the two terminal ends of bones to be fused (e.g., an end of an intermediate phalanx and an end of a distal phalanx). Although such techniques can work relatively well, the open surgical procedure is relatively costly and can result in relatively long recovery times. Accordingly, improved devices and techniques for treating arthritis, such as arthritis of a DIP joint, are desired.

### SUMMARY OF THE DISCLOSURE

Various embodiments of the disclosure relate to medical devices including a joint scrubber, systems including the devices, and methods of using the devices and systems. The devices and systems described herein can be used in the treatment of joints, such as treatment of arthritic joints. For example, exemplary devices and systems are suitable for scrubbing arthritic DIP joints prior to a joint fusion procedure. While the ways in which the various embodiments of the disclosure address the drawbacks of the prior art are discussed in greater detail below, in general, use of the devices and systems described herein can allow treatment of joints, such as DIP joints, and particularly joint fusion or arthrodesis treatment without open surgical resection of the joint to be treated.

In accordance with exemplary embodiments of the disclosure, a medical device for scrubbing a joint includes an arm having a first end, a second end, and a space (or body portion) there between, and a joint scrubber having a first end coupled to the second end of the arm. Exemplary joint scrubbers are configured to break apart or loosen material in a joint, between two adjacent bones—e.g., to facilitate subsequent insertion of another medical device, such as a bone screw. In accordance with various aspects of these embodiments, the joint scrubber includes a compressible element. Exemplary compressible elements increase in outer perimeter in a first direction when a force is applied to the element in a second direction (e.g., along the axis of the arm and/or the element). The joint scrubber can include one or more bending elements that bend (e.g., to increase the diameter or width of the joint scrubber) when a force is applied to the scrubber. The joint scrubber can include one or more slats (e.g., that comprise the bending elements). The slats can be formed by cutting through a width of a sheet of material in a first direction—e.g., parallel the axis of the scrubber or arm. The slats can further include a cut partially through the width in a second direction (e.g., perpendicular or substantially perpendicular the arm or element axis). The joint scrubbers can be formed of any suitable material, such as plastic, or metal, such as stainless steel, titanium, or nitinol. In accordance with various aspects of these embodiments, the joint scrubber comprises deformably resilient material, such that the material can be engaged deform to increase an outer most perimeter and be retracted to decrease the outermost perimeter. The device can include an end cap having a first end attached to a second end of the joint scrubber. A second end of the end cap can be tapered or rounded to facilitate placement of the end cap partially within a bore formed within a bone. The end cap can also include a tapered section between the first end and the second end of the end cap. Dimensions of the arm and scrubber can vary in accordance with an intended application of the device. By way of examples, a length of the arm can range from about 2 cm to about 20 cm, about 5 cm to about 15 cm, about 5 cm to about 10 cm, or be about 8 cm. Outer diameters of the arm and the joint scrubber, when not engaged, are generally configured to fit within a bore formed within a bone corresponding to a joint to be treated. In accordance with exemplary aspects of these embodiments, an outer diameter of the arm is about 0.25 to about 5 mm, 0.5 to about 4 mm, about 1 to about 3 mm, or is about 2 mm; and, an outer diameter of the joint scrubber in an uncompressed position is about 0.25 to about 5 mm, is about 0.5 to about 4 mm, is about 1 to about 3 mm, is about 2 mm. The outer diameter or width of the scrubber when engaged can be about 0.3 to about 8 mm, about 0.5 to about 6 mm, about 2 to about 5 mm. The device can also include a sleeve over a portion of the arm—e.g., proximate the first end of the arm. The device can be configured to be received by a driver, such as an electric driver—e.g., a drill, which can have selectable and/or variable speeds.

In accordance with other exemplary embodiments of the invention, a medical device for scrubbing a joint includes a rigid arm having a first end, a second end, and a space there between, and a joint scrubber having a first end coupled to the second end of the arm and a second end having a diameter or width wider than a diameter of the joint scrubber first end. In accordance with various aspects of these embodiments, the joint scrubber is formed of malleable material that can be non-resiliently deformable. The joint scrubbers can be formed of any suitable material, such as plastic, or metal, such as stainless steel or titanium. In accordance with exemplary aspects of these embodiments, an outer diameter of the arm is about 0.25 to about 5 mm, 0.5 to about 4 mm, about 1 to about 3 mm, or is about 2 mm; and, a width of the second end is about 0.3 to about 8 mm, about 0.5 to about 6 mm, about 2 to about 5 mm, or about 2 mm. A length of the device can range from about 2 cm to about 20 cm, about 5 cm to about 15 cm, about 5 cm to about 10 cm, or be about 8 cm. The second end can also include one or more curved surfaces to facilitate insertion of the device and/or scrubbing of the joint to be treated.

In accordance with yet further exemplary embodiments of the disclosure, a system includes a medical device, such as a medical device as described herein, and a driver, such as an electric device—e.g., a drill.

Both the foregoing summary and the following detailed description are exemplary and explanatory only and are not restrictive of the disclosure.

### BRIEF DESCRIPTION OF THE DRAWINGS

A more complete understanding of exemplary embodiments of the present disclosure may be derived by referring to the detailed description and claims when considered in connection with the following illustrative figures.

FIG. 1 illustrates system including a medical device in accordance with exemplary embodiments of the disclosure.

FIG. 2 illustrates a portion of an exemplary medical device, including a joint scrubber, in accordance with exemplary embodiments of the disclosure.

FIG. 3 illustrates an exemplary joint scrubber in a retracted position in accordance with yet further exemplary embodiments of the disclosure.

FIG. 4 illustrates a joint scrubber in an engaged position in accordance with yet further exemplary embodiments.

FIGS. 5-8 illustrate a method of treating a joint in accordance with yet further exemplary embodiments of the disclosure.

FIGS. 9-11 illustrate another medical device in accordance with additional embodiments of the disclosure.

It will be appreciated that the figures are not necessarily drawn to scale. For example, the dimensions of some of the elements in the figures may be exaggerated relative to other elements to help to improve understanding of illustrated embodiments of the present invention.

### DETAILED DESCRIPTION OF EXEMPLARY EMBODIMENTS

The description of exemplary embodiments of the present invention provided below is merely exemplary and is intended for purposes of illustration only; the following description is not intended to limit the scope of the invention disclosed herein. Moreover, recitation of multiple embodiments having stated features is not intended to exclude other embodiments having additional features or other embodiments incorporating different combinations of the stated features.

The present disclosure describes medical devices and systems that are suitable for use in treatment of joints, and particularly for treatments including joint fusion or arthrodesis, such as arthrodesis of phalanges. For example, exemplary devices and systems described herein are suitable for treatment of a distal interphalangeal (DIP) joint, such as the scrubbing of the joint prior to implantation of another device, such as a bone screw. The devices and systems are conveniently described below in connection with treatment of a DIP joint; however, unless otherwise noted, devices and systems described herein are not limited to such applications.

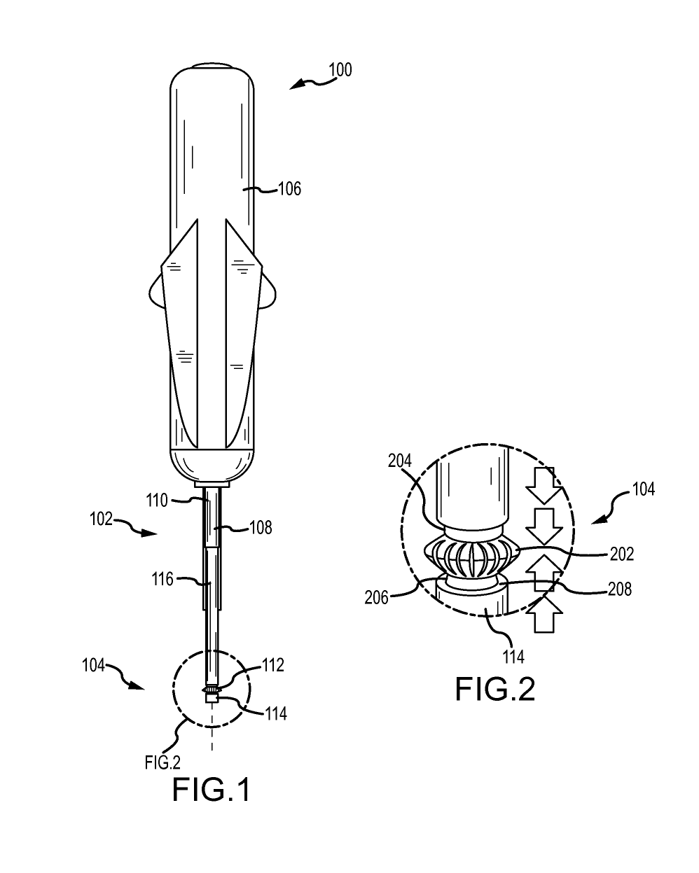

FIG. 1 illustrates a system 100 in accordance with exemplary embodiments of the disclosure. System 100 includes a medical device 102, including a joint scrubber 104 and a driver 106. As set forth in more detail below, during operation, driver 106 can be used to cause device 102 to rotate or to otherwise move to breakup or soften material between two adjacent bones to thereby facilitate insertion of a medical device through the joint.

Exemplary medical device 102 in accordance with various embodiments of the disclosure is illustrated in FIGS. 1-4. Device 102 includes an arm 108, having a first end 110, a second end 112, and a space (or body portion) there between, and joint scrubber 104. Device 102 also includes an end cap 114. First end 110 can be configured to be received by or coupled to driver 106.

Arm 108 may be formed of any suitable material. For example, arm 108 may be formed of any suitable rigid plastic or metal, such as stainless steel, titanium, or the like. Arm 108 can be hollow or a solid rod. If arm 108 is solid, arm 108 can include a recess area to receive joint scrubber 104.

A length and width of arm 108 can vary according to application. By way of examples, a length of the arm 108 is between about 2 cm to about 20 cm, between about 5 cm to about 15 cm, between about 5 cm to about 10 cm, or about 8 cm. An outer diameter (or an equivalent thereof, defined by a maximum width along a cross section of arm 108) is generally smaller than a bore through which device 102 will be inserted. Exemplary widths or diameters of arm 108 can be about 0.25 to about 5 mm, about 0.5 to about 4 mm, about 1 to about 3 mm, or be about 2 mm.

Device 102 can optionally include a sleeve 116 that covers a portion of arm 108. Sleeve 116 may remain stationary while arm 108 moves. Sleeve 116 can be formed of any of the materials described herein in connection with arm 108.

Scrubber 104 is generally configured to breakup or soften material within a joint. Such softening of material can facilitate subsequent procedures on the joint, such as insertion of another device, such as a screw or the like. It may be desirable to treat a joint area that is larger than a cross section of arm 108. In these cases, scrubber 104 can include one or more compressible elements or a bending elements 202 that deforms and causes a perimeter or cross section of scrubber 104 to expand from less than a perimeter or cross section of arm 108 to larger than the perimeter/cross-sectional area of arm 108.

Compressible elements 202 can be in the form of slats, as illustrated. In these cases, scrubber 103 can be formed by forming cuts though material (e.g., material in the configuration of a tube) in a first direction. Scrubber 104 can also include cuts 402 that are partially through the material in a second direction to facilitate operation of scrubber 104. In the illustrated example, the first direction is or substantially is perpendicular the second direction.

In the illustrated example, a force can be applied to scrubber 104 in the directions illustrated by the arrows in FIG. 2. When a force is applied in these directions, joint scrubber 104 expands in a direction that is substantially perpendicular to the axis of arm 108 (which can be the same as the axis of joint scrubber 104). This allows device 102 to “scrub” an area that is larger than the cross-sectional area of arm 108 (e.g., cross-sectional areas that are or that substantially are perpendicular to the axis of arm 108).

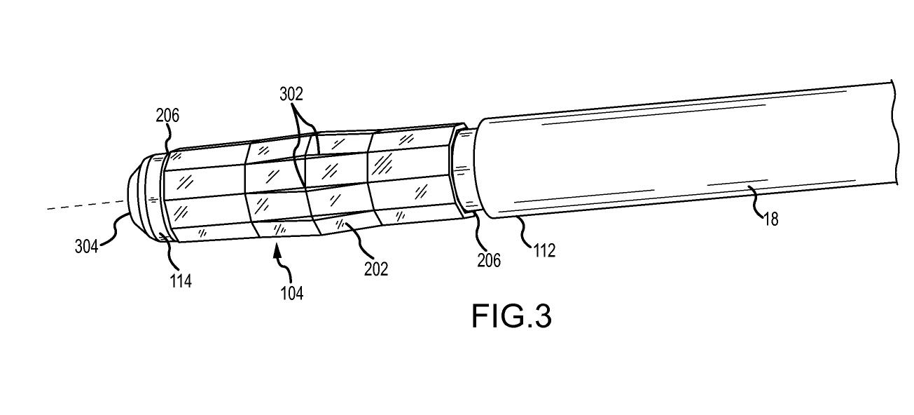

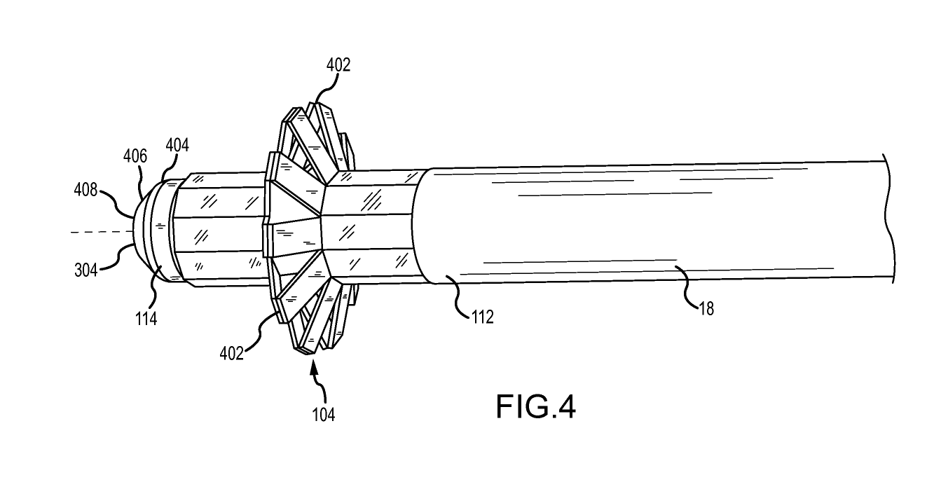

Joint scrubber 104 can be formed of any suitable material. For example, scrubber 104 can be formed of plastic or metal, such as stainless steel, titanium, or nitinol. The material can be deformably resilient, so as to allow scrubber 104 to return to a first state (as illustrated in FIG. 3) after being in a second or expanded state (as illustrated in FIG. 4).

Joint scrubber 104 can include coupling elements 204, 206 to respectively couple joint scrubber 104 to arm 108 and to end cap 114. Coupling elements 204, 206 can be friction fit, snap fit, or be threadedly attached to arm 108 and end cap 114.

As noted above, an outer diameter or width of joint scrubber 104 in an uncompressed or deactivated state (FIG. 3) can be less than the diameter or width of arm 108. By way of examples, the width or diameter of joint scrubber 104 in an uncompressed state can range from about 0.25 to about 5 mm, about 0.5 to about 4 mm, about 1 to about 3 mm, or be about 2 mm. In the compressed or activated state (FIG. 4), an outer diameter or width of joint scrubber 104 can range from about 0.3 to about 8 mm, 0.5 to about 6 mm, or about 2 to about 5 mm.

End cap 114 attaches to a second end or coupling element 106 of joint scrubber 104. Exemplary end cap 114 includes a first end that couples to joint scrubber 104 and a second end 304. End cap 304 can include one or more tapered sections 404, 406, and a rounded or partially spherical end 408. End cap 408 and tapered sections 404 and/or 406 can facilitate placement of cap 114 into a bore formed within a bone (e.g., an intermediate phalanx) and maintaining cap 114 in place as joint scrubber 104 is activated (FIG. 4) and deactivated (FIG. 3).

Turning again to FIG. 1, driver 106 can be any suitable device that causes arm 108 to rotate or otherwise move. Exemplary drivers include variable or single speed electric drill.

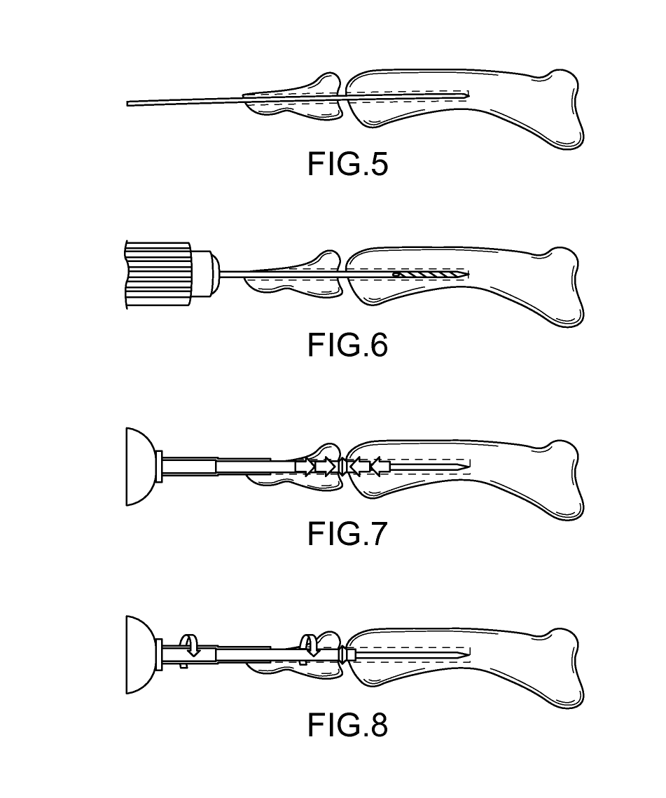

FIGS. 5-8 illustrate a method of using device 100. In the illustrated method, a wire, such as a k-wire is inserted into a tip of a finger—e.g., though the tip of a distal phalanx. Next, a bore is drilled into two adjacent bones—e.g., a distal phalanx and an adjacent intermediate phalanx. Next, device 100 is inserted into the bore, such that joint scrubber 104 is disposed between the adjacent bones. Device 102 is then activated (compressed) and deactivated (uncompressed) to soften or breakup material between the adjacent bones. Device 102 can then be removed—e.g., by reversing a direction of arm 108 used during the drilling step.

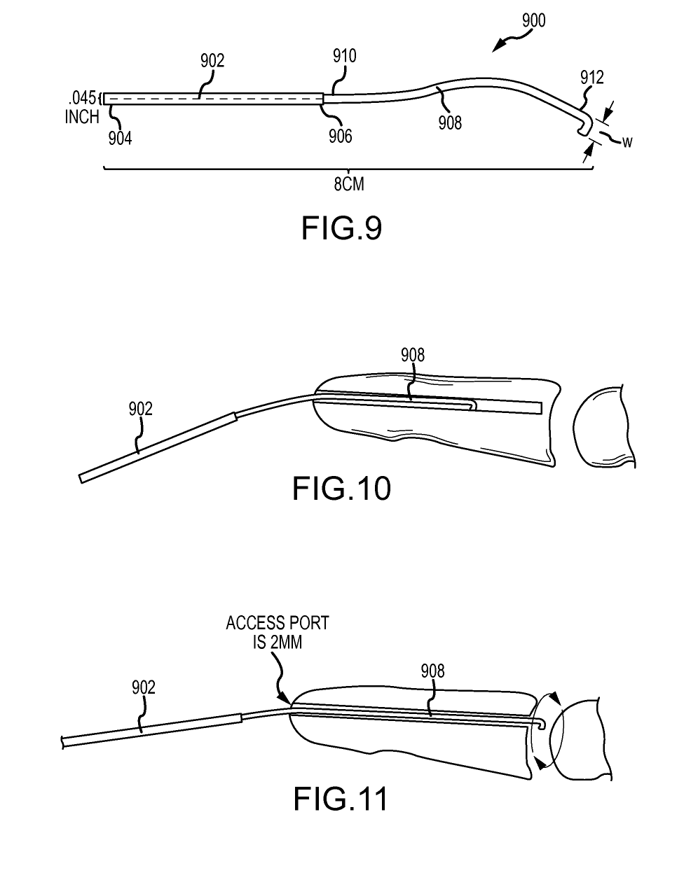

FIGS. 9-11 illustrate another medical device 900 in accordance with additional embodiments of the invention. Joint scrubber 900 includes a rigid arm 902 having a first end 904, a second end 906, and an axis spanning there between; and a joint scrubber 908 having a first end 910 coupled to the second end 906 of the arm 902 and a second end 912 having a width W wider than a diameter of the joint scrubber first end.

Arm 902 can be formed of any of the materials described above in connection with arm 108 and can have the same dimensions as arm 108.

Joint scrubber can be formed of, for example, non-resiliently deformable material such as plastic or metal, such as stainless steel, titanium, or the like. A width of second end can be, for example, greater than a width of arm 902, such as, about 0.3 to about 8 mm, about 0.5 to about 6 mm, about 2 to about 5 mm, or about mm.

An overall length of device 900 can be about 2 cm to about 20 cm, about 5 cm to about 15 cm, about 5 cm to about 10 cm, or about 8 cm.

Device 900 can be used with a driver, such as driver 106 to form a system.

Some specific examples of the invention are described below:

1. A medical device for scrubbing a joint, the device comprising:
an arm having a first end, a second end, and a space there between; and
a joint scrubber having a first end coupled to the second end of the arm.

2. The medical device for scrubbing a joint of example 1, wherein the joint scrubber comprises a compressible element.

3. The medical device for scrubbing a joint of example 2, wherein the compressible element comprises an outer perimeter, and wherein the outer perimeter expands when a force is applied to the compressible element.

4. The medical device for scrubbing a joint of example 3, wherein the force is applied along the axis.

5. The medical device for scrubbing a joint of any of examples 1-4, wherein the joint scrubber comprises at least one bending element.

6. The medical device for scrubbing a joint of any of examples 1-5, wherein the joint scrubber comprises a plurality of bending elements.

7. The medical device for scrubbing a joint of any of examples 1-6, wherein the at least one bending element comprises a slat.

8. The medical device for scrubbing a joint of any of examples 1-6, wherein the joint scrubber comprises a plurality of slats.

9. The medical device for scrubbing a joint of any of examples 1-8, wherein the joint scrubber comprises a plurality of slats formed by forming cuts through material used to form the joint scrubber in a first direction.

10. The medical device for scrubbing a joint of example 9, wherein the plurality of slats further comprise a cut partially through the material in a second direction.

11. The medical device for scrubbing a joint of any of examples 1-10, wherein the joint scrubber comprises plastic.

12. The medical device for scrubbing a joint of any of examples 1-11, wherein the joint scrubber comprises metal.

13. The medical device for scrubbing a joint of example 9, wherein the metal comprises stainless steel.

14. The medical device for scrubbing a joint of example 9, wherein the metal comprises nitinol.

14. The medical device for scrubbing a joint of example 9, wherein the metal comprises titanium.

15. The medical device for scrubbing a joint of any of examples 1-14, wherein the joint scrubber comprises deformably resilient material.

16. The medical device for scrubbing a joint of any of examples 1-14, wherein the device further comprises an end cap coupled to a second end of the joint scrubber.

17. The medical device for scrubbing a joint of example 16, wherein the end cap comprises a first end coupled to the second end of the joint scrubber and a second end.

18. The medical device for scrubbing a joint of example 16, wherein the end cap comprises a first end coupled to the second end of the joint scrubber, a second end, and a tapered section there between.

19. The medical device for scrubbing a joint of any of examples 16-18, wherein a second end of the end cap is configured to reside within a bore formed within an phalanx.

20. The medical device for scrubbing a joint of any of examples 1-19, wherein a length of the arm is between about 2 cm to about 20 cm.

21. The medical device for scrubbing a joint of any of examples 1-20, wherein a length of the arm is between about 5 cm to about 15 cm.

22. The medical device for scrubbing a joint of any of examples 1-21, wherein a length of the arm is between about 5 cm to about 10 cm.

23. The medical device for scrubbing a joint of any of examples 1-22, wherein a length of the arm is about 8 cm.

24. The medical device for scrubbing a joint of any of examples 1-23, wherein an outer diameter of the arm is about 0.25 to about 5 mm.

25. The medical device for scrubbing a joint of any of examples 1-24, wherein an outer diameter of the arm is about 0.5 to about 4 mm.

26. The medical device for scrubbing a joint of any of examples 1-25, wherein an outer diameter of the arm is about 1 to about 3 mm.

27. The medical device for scrubbing a joint of any of examples 1-25, wherein an outer diameter of the arm is about 2 mm.

28. The medical device for scrubbing a joint of any of examples 1-27, wherein an outer diameter of the joint scrubber in an uncompressed position is less than the outer diameter of the arm.

29. The medical device for scrubbing a joint of any of examples 1-28, wherein an outer diameter of the joint scrubber in an uncompressed position is about 0.25 to about 5 mm.

30. The medical device for scrubbing a joint of any of examples 1-29, wherein an outer diameter of the joint scrubber in an uncompressed position is about 0.5 to about 4 mm.

31. The medical device for scrubbing a joint of any of examples 1-30, wherein an outer diameter of the joint scrubber in an uncompressed position is about 1 to about 3 mm.

32. The medical device for scrubbing a joint of any of examples 1-31, wherein an outer diameter of the joint scrubber in an uncompressed position is about 2 mm.

33. The medical device for scrubbing a joint of any of examples 1-32, wherein an outer diameter of the joint scrubber in a compressed state is about 0.3 to about 8 mm.

34. The medical device for scrubbing a joint of any of examples 1-33, wherein an outer diameter of the joint scrubber in a compressed state is about 0.5 to about 6 mm.

35. The medical device for scrubbing a joint of any of examples 1-33, wherein an outer diameter of the joint scrubber in a compressed state is about 2 to about 5 mm.

36. The medical device for scrubbing a joint of any of examples 1-35, wherein the device further comprises a sleeve about a portion of the arm.

37. The medical device for scrubbing a joint of any of examples 1-36, wherein the first end of the arm is configured to be received by a driver.

37. The medical device for scrubbing a joint of any of examples 1-37, wherein the first end of the arm is configured to be received by a drill.

38. A system comprising a medical device of any of examples 1-37 and a driver.

39. The system of example 38, wherein the device comprises an electric motor.

40. The system of any of examples 38-39, wherein the driver comprises a drill.

41. The system of any of examples 38-40, wherein a rotation speed of the driver is variable.

42. The system of any of examples 38-41, wherein the driver comprises a sleeve configured to receive the medical device.

43. A method of using the device of any of examples 1-37.

44. A method of using the system of any of examples 38 to 42.

45. A medical device for scrubbing a joint, the device comprising:
a rigid arm having a first end, a second end, and a space there between; and
a joint scrubber having a first end coupled to the second end of the arm and a second end having a width wider than a width of the joint scrubber first end.

46. The medical device for scrubbing a joint of example 45, wherein the joint scrubber comprises malleable material.

46. The medical device for scrubbing a joint of example 45, wherein the joint scrubber comprises non-resiliently deformable material.

47. The medical device for scrubbing a joint of any of examples 45-46, wherein an outer diameter of the arm is about 0.25 to about 5 mm.

48. The medical device for scrubbing a joint of any of examples 45-47, wherein an outer diameter of the arm is about 0.5 to about 4 mm.

49. The medical device for scrubbing a joint of any of examples 45-48, wherein an outer diameter of the arm is about 1 to about 3 mm.

50. The medical device for scrubbing a joint of any of examples 45-49, wherein an outer diameter of the arm is about 2 mm.

51. The medical device for scrubbing a joint of any of examples 45-50, wherein a width of the second end is about 0.3 to about 8 mm.

52. The medical device for scrubbing a joint of any of examples 45-51, wherein a width of the second end is about 0.5 to about 6 mm.

53. The medical device for scrubbing a joint of any of examples 45-52, wherein a width of the second end is about 2 to about 5 mm.

54. The medical device for scrubbing a joint of any of examples 45-53, wherein a length of the device is between about 2 cm to about 20 cm.

55. The medical device for scrubbing a joint of any of examples 45-54, wherein a length of the device is between about 5 cm to about 15 cm.

56. The medical device for scrubbing a joint of any of examples 45-55, wherein a length of the device is between about 5 cm to about 10 cm.

57. The medical device for scrubbing a joint of any of examples 45-55, wherein a length of the device is about 8 cm.

58. The medical device for scrubbing a joint of any of examples 45-56, wherein the joint scrubber comprises plastic.

59. The medical device for scrubbing a joint of any of examples 45-57, wherein the joint scrubber comprises metal.

60. The medical device for scrubbing a joint of example 59, wherein the metal comprises stainless steel.

61. The medical device for scrubbing a joint of example 59, wherein the metal comprises stainless titanium.

62. The medical device for scrubbing a joint of any of examples 45-57, wherein the second end comprises a curved surface.

The present invention has been described above with reference to a number of exemplary embodiments and examples. It should be appreciated that the particular embodiments shown and described herein are illustrative of the preferred embodiments of the invention and its best mode, and are not intended to limit the scope of the invention as set forth in the claims. It will be recognized that changes and modifications may be made to the embodiments described herein without departing from the scope of the present invention. These and other changes or modifications are intended to be included within the scope of the present invention, as expressed in the following claims and the legal equivalents thereof

What is claimed is:

1. A medical device for scrubbing a joint, the device comprising:
an arm having a first end, a second end, and a space there between; and
a joint scrubber having a first end coupled to the second end of the arm.

2. The medical device for scrubbing a joint of claim 1, wherein the joint scrubber comprises a compressible element.

3. The medical device for scrubbing a joint of claim 2, wherein the compressible element comprises an outer perimeter, and wherein the outer perimeter expands when a force is applied to the compressible element.

4. The medical device for scrubbing a joint of claim 3, wherein the force is applied along the axis.

5. The medical device for scrubbing a joint of claim 1, wherein the joint scrubber comprises at least one bending element.

6. The medical device for scrubbing a joint of claim 1, wherein the joint scrubber comprises a plurality of bending elements.

7. The medical device for scrubbing a joint of claim 1, wherein at least one bending element comprises a slat.

8. The medical device for scrubbing a joint of claim 1, wherein the joint scrubber comprises a plurality of slats.

9. The medical device for scrubbing a joint of claim 1, wherein the joint scrubber comprises a plurality of slats formed by forming cuts through material used to form the joint scrubber in a first direction.

10. The medical device for scrubbing a joint of claim 9, wherein the plurality of slats further comprise a cut partially through the material in a second direction.

11. The medical device for scrubbing a joint of claim 1, wherein the joint scrubber comprises plastic.

12. The medical device for scrubbing a joint of claim 1, wherein the joint scrubber comprises metal.

13. The medical device for scrubbing a joint of claim 1, wherein the joint scrubber comprises deformably resilient material.

14. The medical device for scrubbing a joint of claim 1, wherein the device further comprises an end cap coupled to a second end of the joint scrubber.

15. The medical device for scrubbing a joint of claim 14, wherein the end cap comprises a first end coupled to the second end of the joint scrubber and a second end.

16. The medical device for scrubbing a joint of claim 14, wherein the end cap comprises a first end coupled to the second end of the joint scrubber, a second end, and a tapered section there between.

17. The medical device for scrubbing a joint of claim 14, wherein the second end of the end cap is configured to reside within a bore formed within an phalanx.

18. The medical device for scrubbing a joint of claim 1, wherein a length of the arm is between about 2 cm to about 20 cm.

19. The medical device for scrubbing a joint of claim 1, wherein an outer diameter of the joint scrubber in an uncompressed position is less than the outer diameter of the arm.

20. The medical device for scrubbing a joint of claim 1, wherein an outer diameter of the joint scrubber in an uncompressed position is about 0.25 to about 5 mm.
